(19) United States
(12) Patent Application Publication
(10) Pub. No.: US 2025/0375124 A1
(43) Pub. Date: Dec. 11, 2025

(54) WIRELESS RESPIRATORY MONITORING DEVICE

(71) Applicant: SEOUL NATIONAL UNIVERSITY R&DB FOUNDATION, Seoul (KR)

(72) Inventors: Gyuchul Yi, Seoul (KR); Yunjae Hwang, Seoul (KR); Kyoungho Kim, Incheon (KR)

(21) Appl. No.: 18/686,787

(22) PCT Filed: Mar. 11, 2022
(86) PCT No.: PCT/KR2022/003434

§ 371 Date: Jan. 28, 2025

(30) Foreign Application Priority Data
Sep. 2, 2021 (KR) 10-2021-0117244

(51) Int. Cl. A61B 5/087; A61B 5/00

(52) U.S. Cl. CPC A61B 5/087; A61B 5/6819; A61B 2560/0214; A61B 2560/0468; A61B 2562/0285; A61B 2562/227

(57) ABSTRACT

The present disclosure provides a wireless respiratory monitoring device including a main body unit including a middle body portion having an insertion portion inserted into a user's nose, and an extended body portion extending from the middle body portion to both sides and configured to surround and hold a nosewing portion of the user, and a piezoresistive sensor which is installed on an inner surface of the insertion portion and disposed inside the user's nose, includes a plurality of nanorods having a piezoresistive property, and is configured to sense the user's breathing characteristics by using resistance changes caused by deformation of the nanorods according to airflow changes due to the user's breathing.

### TECHNICAL FIELD

The present invention relates to health and diagnostic devices, and more particularly, to sensors and monitoring devices related to breathing.

### BACKGROUND ART

When diagnosing sleep apnea, apnea is generally defined as a decrease in respiratory airflow of 90% or more for more than 10 seconds. To diagnose sleep apnea, respiratory airflow must be measured. Respiratory data must be measured stably from involuntary body movements or the influence of the external environment during sleeping, and the measuring device must also be firmly maintained at the measurement location. In addition, the respiratory measurement device must not be so uncomfortable that it interferes with sleep.

Existing respiratory monitoring devices are employing a method for directly measuring airflow in the vicinity of the respiratory organ and a method of indirectly estimating it through other biological signals. In the method for indirectly estimating respiratory airflow, the pressure or movement of the chest or abdomen caused by respiratory is measured by using a device mounted on the chest or abdomen, or the sound of breathing is measured and it is used to estimate respiratory airflow. This method has a problem in that measurement data may be easily distorted due to body movement or the external environment. Meanwhile, there are two ways to directly measure respiratory airflow: one is to measure respiratory airflow by making respiratory airflow independent from the external environment by using a device such as a tube, and the other is to have the measuring device outside the respiratory organ. The method in which the measuring device is outside the respiratory organ measures respiratory airflow by attaching a sensor or flow meter around the nose or mouth. However, in this case, since the sensor is exposed to the outside of the respiratory organ (tract), there is a possibility that the data may be distorted by body movement or external air currents.

Furthermore, when using a wired measuring device, the problems such as tangled strings may occur due to tossing and turning during sleep, and sleeping may be disrupted by the string getting caught on the body. There is also a method that uses a mask-type device to achieve independent respiratory airflow and embeds sensors and circuits in the mask, but in this case, since the mask device covers a most area of the face, the wearer may feel uncomfortable and have difficulty in sleeping.

### DISCLOSURE OF THE INVENTION

### Technical Problem

The technological object to be achieved by the present invention is to provide a wireless respiratory monitoring device which may be used for the diagnosis of sleep apnea, etc., and where a sensor is placed inside the respiratory organ (tract) to measure respiratory airflow independently from the external environment or external airflow, while at the same time feeling almost no inconvenience even when worn while sleeping.

The objects to be achieved by the present invention are not limited to the objects mentioned above, and other objects not mentioned will be understood by those skilled in the art from the description below.

### Technical Solution

According to one embodiment of the present invention, there is provided a wireless respiratory monitoring device comprising: a main body unit including a middle body portion having an insertion portion inserted into a user's nose, and an extended body portion extending from the middle body portion to both sides and configured to surround and hold a nosewing portion of the user; a piezoresistive sensor which is installed on an inner surface of the insertion portion and disposed inside the user's nose, includes a plurality of nanorods having a piezoresistive property, and is configured to sense the user's breathing characteristics by using resistance changes caused by deformation of the nanorods according to airflow changes due to the user's breathing; a circuit unit disposed within the extended body portion and electrically connected to the piezoresistive sensor; a wireless communication unit disposed within the extended body portion and electrically connected to the circuit unit; and a battery member disposed within the extended body portion and configured to supply power to the circuit unit and the wireless communication unit.

The insertion portion may include a first cylindrical tube and a second cylindrical tube corresponding to the user's two nostrils.

The piezoresistive sensor may include a first piezoresistive sensor installed on an inner side of the first cylindrical tube and a second piezoresistive sensor installed on an inner side of the second cylindrical tube.

The piezoresistive sensor may includes a first electrode; the plurality of nanorods disposed on the first electrode and substantially perpendicular to the first electrode; an insulating layer disposed on the first electrode to bury the plurality of nanorods from a lower portion thereof to a certain height; and a second electrode disposed on the insulating layer to contact the plurality of nanorods. The plurality of nanorods may include a protruding region which protrudes above the insulating layer, and the protruding region of the plurality of nanorods may be configured to be deformed according to a change in airflow due to the user's breathing.

A diameter of the plurality of nanorods may be greater than or equal to about 100 nm and less than about 1 μm, a thickness of the insulating layer may be approximately 1 μm to 3 μm, and a length of the protruding region of the plurality of nanorods may be approximately 7 μm to 14 μm.

The plurality of nanorods may include at least one of silicon (Si) or zinc oxide (Zn oxide).

The circuit unit may include a constant voltage generating circuit for applying a constant voltage to the piezoresistive sensor; and a current measurement circuit for measuring a change in current of the piezoresistive sensor according to a change in resistance of the plurality of nanorods.

The current measurement circuit may include a sensing resistor, an amplifier, and an analog-to-digital converter. The sensing resistor may be connected to the piezoresistive sensor, the analog-to-digital converter may be connected to the wireless communication unit, and the amplifier may be connected between the sensing resistor and the analog-to-digital converter.

The wireless communication unit may include a Bluetooth module or a wireless LAN module.

The extended body portion may include a first extended body portion extending from one end of the middle body portion and a second extended body portion extending from another end of the middle body portion, and the battery member may include a first battery member disposed within the first extended body portion and a second battery member disposed within the second extended body portion.

At least a portion of the extended body portion and the middle body portion may be made of a flexible material.

### Advantageous Effects

According to embodiments of the present invention, it is possible to implement a wireless respiratory monitoring device which may be used for the diagnosis of sleep apnea, etc., and has a sensor placed inside the respiratory organ (tract) to measure respiratory airflow independently from the external environment (external airflow), while causing little discomfort when worn while sleeping.

Most existing respiratory monitoring devices measure respiratory airflow outside the respiratory organ or estimate the amount of respiratory through other biological signals. This method has the problem that measurement data may be easily distorted by the external environment (external airflow). Meanwhile, the devices for monitoring respiratory independently from the external environment covers the face as form of a mask, which may cause discomfort to the wearer while sleeping.

However, as the wireless respiratory monitoring device according to an embodiment of the present invention measures respiratory airflow inside the nose by using an ultra-small piezoresistive sensor and operates wirelessly with an embedded battery and wireless communication module, it may monitor breathing independently and stably from the external environment (external airflow) without causing discomfort to the wearer. Thus, it may be effectively used to diagnose sleep apnea, and the like. In particular, since the piezoresistive sensor includes a plurality of nanorods having a piezoresistive property, and is configured to sense the wearer's respiratory characteristics by using the change in resistance due to deformation of the nanorods according to the change in airflow due to the wearer's (user's) breathing, excellent sensing characteristics may be secured.

### BRIEF DESCRIPTION OF THE DRAWINGS

FIG. 1 is a diagram for explaining a wireless respiratory monitoring device according to an embodiment of the present invention.

FIG. 2 is a perspective diagram illustrating an example of a wireless respiratory monitoring device according to an embodiment of the present invention.

FIG. 3 is a cross-sectional diagram showing a piezoresistive sensor which may be applied to a wireless respiratory monitoring device according to an embodiment of the present invention.

FIG. 4 is a cross-sectional diagram for explaining a sensing method of a piezoresistive sensor that may be applied to a wireless respiratory monitoring device according to an embodiment of the present invention.

FIG. 5 is a graph showing the results obtained by measuring respiration characteristics by using a piezoresistive sensor which may be applied to a wireless respiration monitoring device according to an embodiment of the present invention.

FIG. 6 is a diagram schematically showing experimental equipment for checking sensing accuracy and sensitivity of a piezoresistive sensor which may be applied to a wireless respiratory monitoring device according to an embodiment of the present invention.

FIG. 7 is a graph showing the results obtained by measuring the current change of a piezoresistive sensor while changing the flow rate of gas discharged toward the piezoresistive sensor which may be applied to the wireless respiratory monitoring device according to an embodiment of the present invention.

FIG. 8 is a block diagram showing a circuit configuration which may be applied to a wireless respiratory monitoring device according to an embodiment of the present invention.

### BEST MODE FOR CARRYING OUT THE INVENTION

Hereinafter, embodiments of the present invention will be described in detail with reference to the accompanying drawings.

The embodiments of the present invention to be described below are provided to more clearly explain the present invention to those skilled in the art, and the scope of the present invention is not limited by the following embodiments, and the embodiments may be modified in many different forms.

The terms used in this specification are used to describe specific embodiments and are not intended to limit the present invention. The terms indicating a singular form used herein may include plural forms unless the context clearly indicates otherwise. Also, as used herein, the terms, “comprise” and/or “comprising” specify the presence of the stated shape, step, number, operation, member, element, and/or group thereof and does not exclude the presence or addition of one or more other shapes, steps, numbers, operations, elements, elements and/or groups thereof. In addition, the term, “connection” used in this specification means not only a direct connection of certain members, but also a concept including an indirect connection in which other members are interposed between the members.

In addition, in the present specification, when a member is said to be located “on” another member, this arrangement includes not only a case in which a member is in contact with another member, but also a case where another member exists between the two members. As used herein, the term, “and/or” includes any one and all combinations of one or more of the listed items. In addition, the terms of degree such as “about” and “substantially” used in the present specification are used as a range of values or degrees, or as a meaning close thereto, taking into account inherent manufacturing and substance tolerances, and exact or absolute figures provided to aid in the understanding of this application are used to prevent the infringers from unfairly exploiting the stated disclosure.

Hereinafter, embodiments of the present invention will be described in detail with reference to the accompanying drawings. A size or a thickness of areas or parts shown in the accompanying drawings may be slightly exaggerated for clarity of the specification and convenience of description. The same reference numbers indicate the same configuring elements throughout the detailed description.

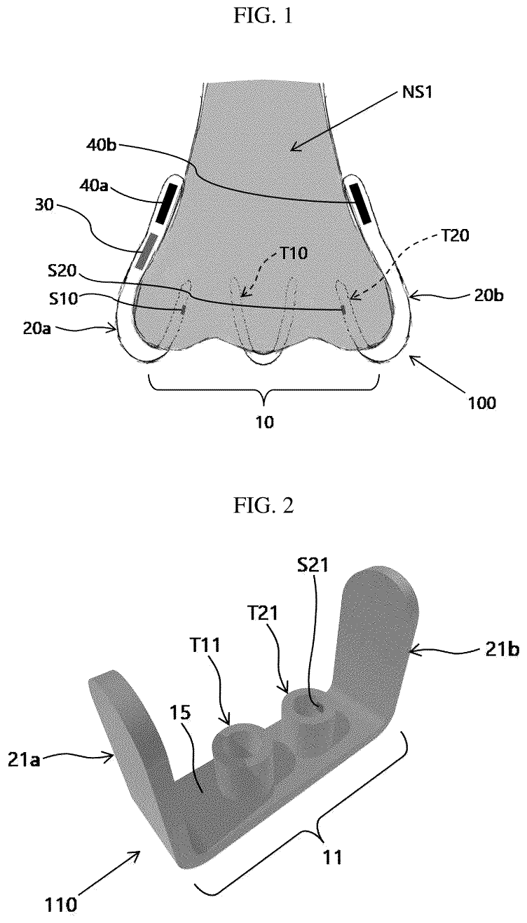

FIG. 1 is a diagram for explaining a wireless respiratory monitoring device according to an embodiment of the present invention.

Referring to FIG. 1, the wireless respiratory monitoring device according to an embodiment of the present invention may include a main body unit 100. The main body unit 100 may include a middle body portion 10 having an insertion portion inserted into the user's nose NS1, and may include extended body portions 20a and 20b configured to extend from the middle body portion 10 to both sides to surround and hold a nosewing portion (nasal ala: rounded side of the nose) of the user.

For example, the insertion portion may include first and second cylindrical tubes T10 and T20 corresponding to the user's two nostrils, respectively. The first and the second cylindrical tubes T10 and T20 may be inserted and placed into the user's two nostrils, respectively. The middle body portion 10 may further include a ‘middle support portion’ on which the first and the second cylindrical tubes T10, T20 are supported, and the first and the second cylindrical tubes T10, T20 may be formed on the middle support portion. At least a portion of the middle body portion 10 may be made of a flexible material.

The extended body portions 20a and 20b may include a first extended body portion 20a extending from one end of the middle body portion 10 and a second extended body portion 20b extending from another end of the middle body portion 10. At least a portion of the extended body portions 20a and 20b may be made of a flexible material. The extended body portions 20a and 20b may serve to fix the wireless respiratory monitoring device to the user's nose NS1 by wrapping and holding the nosewing portion of the user with appropriate tension. The first extended body portion 20a may be disposed while outwardly surrounding one nosewing portion, and the second extended body portion 20b may be disposed while outwardly surrounding the other nosewing portion. Since the first and second cylindrical tubes T10, T20 are inserted into the user's nose NS1, and the first and second extended body portions 20a, 20b surround and hold the user's nosewing portion, the wireless respiratory monitoring device according to the embodiment of the present invention may be stably fixed to the user's nose NS1, may stably maintain its position even while sleeping, and may not cause enormous inconvenience to a wearer (a user). Furthermore, since the tubes T10 and T20 may have a flexible structure with a thin thickness, foreign body sensation may be minimized when worn.

Each of the extended body portions 20a and 20b may extend to a height of about ⅖ to ¾ of the nose NS1 while surrounding the user's nosewing portion. Also, each of the extended body portions 20a and 20b may have a length of approximately 1.5 to 3 times the length of the cylindrical tubes T10 and T20.

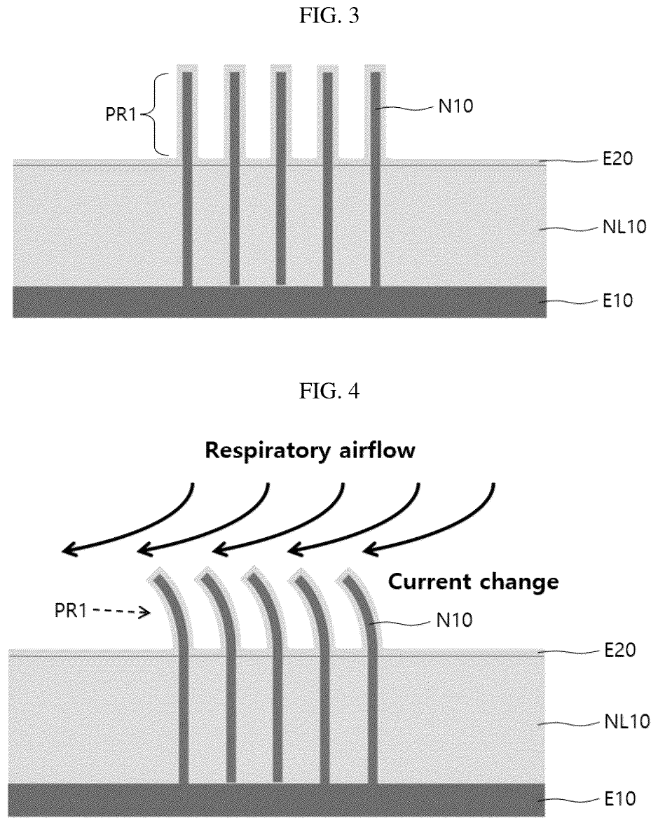

The wireless respiratory monitoring device according to an embodiment of the present invention may include piezoresistive sensors S10, S20 installed on an inner surface of the insertion portion (i.e., T10, T20) and disposed inside the user's nose NS1. The piezoresistive sensors S10, S20 may include a plurality of nanorods (or nanowires) having a piezoresistive property, and may be configured to sense the user's breathing characteristics by using resistance change (changes in resistance of nanorods) due to deformation of the nanorods according to airflow changes due to the user's breathing. The configuration and the principle of the piezoresistive sensors S10, S20 will be described in more detail later with reference to FIGS. 3 and 4.

The piezoresistive sensors S10, S20 may include a first piezoresistive sensor S10 installed on an inner side of the first cylindrical tube T10 and a second piezoresistive sensor S20 installed on an inner side of the second cylindrical tube T20. In this case, the first piezoresistive sensor S10 may be placed at a position close to one side of the nosewing portion (left nosewing portion in the drawing) rather than the central portion of the nose NS1, and the second piezoresistive sensor S20 may be placed at a position close to the other nosewing portion (right nosewing portion in the drawing) rather than the central portion of the nose NS1. Accordingly, the first and second piezoresistive sensors S10 and S20 may be arranged symmetrically left and right with respect to the center portion of the nose NS1. When the first and second piezoresistive sensors S10, S20 are arranged in this way, the changes in airflow due to respiratory may be measured more easily and effectively. However, the positions where the first and second piezoresistive sensors S10 and S20 are placed may change depending on the case.

Meanwhile, the piezoresistive sensors S10 and S20 may be placed at a position which is at least a few mm away from (into) the entrance of the nostril (nose hole). For example, the piezoresistive sensors S10 and S20 may be disposed at a position approximately 3 mm or more away from (into) the entrance of the nostril (nose hole). The gap between each piezoresistive sensor (S10, S20) and the entrance of the corresponding nostril (nose hole) may be, for example, about 3 mm to 15 mm.

In addition, although not shown in FIG. 1, a ‘breathable protective cover’ may be further provided to protect each of the piezoresistive sensors S10 and S20. The breathable protective cover may serve to protect the piezoresistive sensors S10, S20 without interfering with respiratory airflow passing next to or around the piezoresistive sensors S10, S20.

The wireless respiratory monitoring device according to an embodiment of the present invention may include a circuit and communication unit 30 disposed within the extended body portion 20a and/or 20b, and in addition, it may include battery members 40a and 40b disposed within the extended body portion 20a and/or 20b. The circuit and communication unit 30 may be disposed within at least one of the first and second extended body portions 20a and 20b, for example, the first extended body portion 20a. The battery members 40a and 40b may include, for example, a first battery member 40a disposed within the first extended body portion 20a and a second battery member 40b disposed within the second extended body portion 20b. The first and second battery members 40a and 40b may be disposed adjacent to ends of the first and second extended body portions 20a and 20b, respectively. However, the number or the formation position of the circuit and communication unit 30 and the battery members 40a and 40b may vary depending on the case.

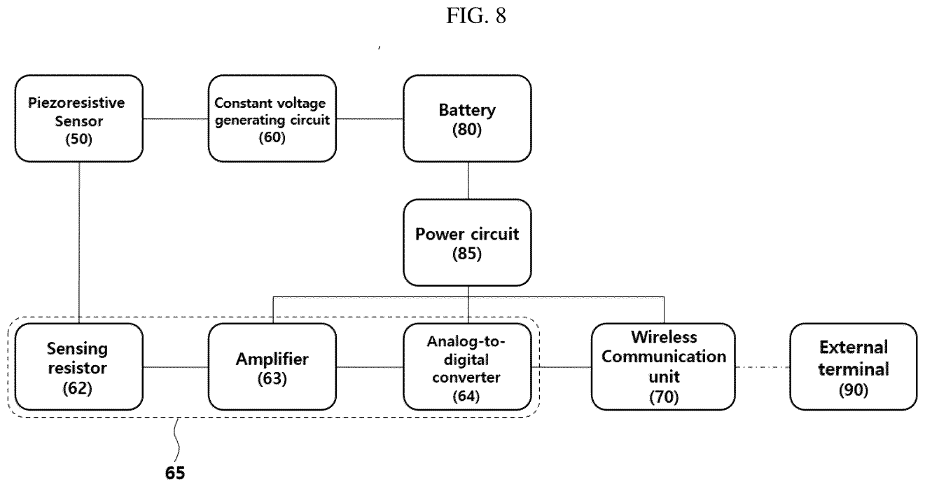

The circuit and communication unit 30 may include a ‘circuit unit’ which is electrically connected to the piezoresistive sensors S10, S20 and performs driving and measurement on the piezoresistive sensors S10, S20, and a ‘wireless communication unit’ which is electrically connected to the circuit unit and performs wireless communication with an external device. The circuit unit and the wireless communication unit will be described in detail later with reference to FIG. 8. The battery members 40a and 40b may serve to supply power to the circuit unit and the wireless communication unit. As the battery members 40a and 40b, for example, a small rechargeable lithium ion battery, a small rechargeable lithium polymer battery, or a disposable mercury battery may be used.

FIG. 2 is a perspective diagram illustrating an example of a wireless respiratory monitoring device according to an embodiment of the present invention.

Referring to FIG. 2, the wireless respiratory monitoring device according to an embodiment of the present invention may include a main body unit 110. The main body unit 110 may have a type of band shape. The main body unit 110 may include a middle body portion 11 having an insertion portion inserted into the user's nose, and an extended body portion 21a, 21b configured to extend from the middle body portion 11 to both sides to surround and hold a nosewing portion of the user. For example, the insertion portion may include first and second cylindrical tubes T11 and T21 respectively corresponding to the user's two nostrils. The first and second cylindrical tubes T11 and T21 may be inserted and placed into the user's two nostrils, respectively. The middle body portion 11 may include a middle support portion 15 on which the first and second cylindrical tubes T11 and T21 are supported, and the first and second cylindrical tubes T11 and T21 spaced apart from each other may be disposed on the middle support portion 15. A through-hole may be formed at positions corresponding to the first and second cylindrical tubes T11 and T21 of the middle support portion 15. The middle support portion 15 may be referred to as a ‘middle band portion’.

The extended body portions 21a and 21b may include a first extended body portion 21a extending from one end of the middle body portion 11 and a second extended body portion 21b extending from another end of the middle body portion 11. The extended body portions 21a and 21b may be referred to as an ‘extension band portion’ or an ‘extension strap portion’. The extended body portions 21a and 21b may serve to fix the wireless respiratory monitoring device to the user's nosewing portion by wrapping and holding the user's nosewing portion with appropriate tension.

At least a portion of the middle support portion 15, the cylindrical tubes T11 and T21, and the extended body portions 21a and 21b may be made of a flexible material. For example, the middle support portion 15, the cylindrical tubes T11 and T21, and the extended body portions 21a and 21b may be made of a polymer such as silicone.

The wireless respiratory monitoring device may include a piezoresistive sensor S21 installed on the inner surface of the insertion portion (i.e., T11, T21) and disposed inside the user's nose. The piezoresistive sensor S21 as shown here is a ‘second piezoresistive sensor’ installed on the inner surface of the second cylindrical tube T21. Although not shown in FIG. 2, a ‘first piezoresistive sensor’ (corresponding to S10 in FIG. 1) may be installed on the inner surface of the first cylindrical tube T11. For convenience, the first piezoresistive sensor installed on the inner surface of the first cylindrical tube T11 in FIG. 2 is referred to as S11. The piezoresistive sensors S11 and S21 may include a plurality of nanorods having a piezoresistive property, and may be configured to sense the user's breathing characteristics by using resistance changes (a resistance change of the nanorods) due to deformation of the nanorods according to airflow changes due to the user's breathing.

In addition, although not shown in FIG. 2, the wireless respiratory monitoring device may include a ‘circuit and communication unit’ (corresponding to 30 in FIG. 1) disposed within the extended body portion (21a and/or 21b), and may further include a ‘battery member’ (corresponding to 40a and 40b in FIG. 1) disposed within the extended body portion (21a and/or 21b). The circuit and communication unit may include a ‘circuit unit’ which is electrically connected to the piezoresistive sensors S11, S21 and performs driving and measurement on the piezoresistive sensors S11, S21, and a ‘wireless communication unit’ which is electrically connected to the circuit unit and performs wireless communication with an external device. The battery member may serve to supply power to the circuit unit and the wireless communication unit.

The wireless respiratory monitoring device as described in FIGS. 1 and 2 may be a type of wearable device. The wireless respiratory monitoring device can be said to be a nasal insertable wearable device that may insert a part of it into the nose. Since the wireless respiratory monitoring device has a small and lightweight structure which may be worn on the human body, discomfort generated when worn may be minimized.

FIG. 3 is a cross-sectional diagram showing a piezoresistive sensor which may be applied to a wireless respiratory monitoring device according to an embodiment of the present invention.

Referring to FIG. 3, the piezoresistive sensor which may be applied to the wireless respiratory monitoring device according to an embodiment of the present invention may include a first electrode E10 and a plurality of nanorods N10 disposed on the first electrode E10. The first electrode E10 may have a plate-shaped structure and may be a conductive substrate or an electrode layer formed on a certain substrate. The first electrode E10 may be formed of a metal, a metal compound, or a conductive polymer.

The plurality of nanorods N10 may be disposed substantially perpendicular to the first electrode E10 on the first electrode E10. The plurality of nanorods N10 may be arranged, for example, in an array having a plurality of rows and a plurality of columns. The plurality of nanorods N10 may be made of a material having a piezoresistive property. For example, the plurality of nanorods N10 may include at least one material selected from silicon (Si) and zinc oxide (Zn oxide). The plurality of nanorods N10 may be easily formed by using an etching process or a growth process. Since the nanorod N10 have a piezoresistive property, when the shape of the nanorod N10 is changed due to a pressure, the electrical resistance of the nanorod N10 changes, and as a result, the current passing through the nanorod N10 may change. The degree of change in current may vary depending on the degree of deformation of the nanorod N10. Therefore, the change in a pressure due to the respiratory airflow applied to the nanorod N10 may be measured by measuring the change in current flowing through the nanorod N10.

The diameter of each of the plurality of nanorods N10 may be greater than or equal to about 100 nm and less than about 1 μm. The gap between the plurality of nanorods N10 may be about 5 μm to about 10 μm. The length (height) of the plurality of nanorods N10 may be about 10 μm to 15 μm. When these conditions are satisfied, measurement (sensing) of respiratory airflow using the plurality of nanorods N10 may be performed more easily.

The piezoresistive sensor may include an insulating layer NL10 disposed on the first electrode E10 to bury the plurality of nanorods N10 from a lower portion thereof to a certain height, and a second electrode E20 disposed to contact with the plurality of nanorods N10 on the insulating layer NL10. The insulating layer NL 10 may be formed to fill the space between the plurality of nanorods N10 and bury the plurality of nanorods N10 to a certain height. For example, the insulating layer NL10 may be formed of an insulating polymer such as polyimide or may be made of various other insulating materials. In some cases, at least a portion of the insulating layer NL10 may be composed of an air layer (a type of insulator). The thickness of the insulating layer NL10 may be, for example, about 1 μm to 3 μm. The second electrode E20 may be conformally formed along the surface of the insulating layer NL10 and the exposed surfaces of the plurality of nanorods N10. The second electrode E20 may be formed to have a fairly thin thickness. For example, the second electrode E20 may be formed to have a thickness of about 100 nm or less. The second electrode E20 may be formed by a flexible conductive material. The portion of the second electrode E20 formed on the nanorods N10 may be easily changed together with the nanorods N10. The insulating layer NL10 may serve to electrically separate the first electrode E10 and the second electrode E20.

The plurality of nanorods N10 may have a protruding region PRI which protrudes above the insulating layer NL10, and the protruding region PRI of the plurality of nanorods N10 may be deformed according to a change in airflow due to the user's breathing. The length of the protruding region PRI of the plurality of nanorods N10 may be about 7 μm to 14 μm. When this condition is satisfied, measurement sensing of respiratory airflow using the plurality of nanorods N10 may be performed more effectively. As the nanorod N10 is deformed by the respiratory airflow, the magnitude of the current flowing between the first electrode E10 and the second electrode E20 may change.

The piezoresistive sensor may be a type of ‘pressure sensor’. The piezoresistive sensor may be manufactured to have a width, a height, and a thickness of about 5 mm or less, about 3 mm or less, or about 1 mm or less. Therefore, the piezoresistive sensor may be said to be an ultra-small pressure sensor.

FIG. 4 is a cross-sectional diagram for explaining a sensing method of a piezoresistive sensor which may be applied to a wireless respiratory monitoring device according to an embodiment of the present invention.

Referring to FIG. 4, the plurality of nanorods N10 according to an embodiment of the present invention may have the protruding region PRI protruding above the insulating layer NL10, and the protruding region PRI of the plurality of nanorods N10 may be deformed according to changes in airflow caused by the user's breathing. As the nanorod N10 is deformed by the respiratory airflow, the resistance of the nanorod N10 may change and the magnitude of the current flowing between the first electrode E10 and the second electrode E20 may change. When a constant voltage is applied between the first electrode E10 and the second electrode E20, and the nanorods N10 are deformed by the respiratory airflow, the resistance of the nanorods N10 may be change due to the piezoresistive effect, and the magnitude of the current flowing between the first electrode E10 and the second electrode E20 may change. Therefore, the change in pressure due to the respiratory airflow applied to the nanorods N10 may be measured by measuring the change in current flowing through the nanorods N10. That is, breathing characteristics may be monitored by measuring changes in the current of the nanorods N10.

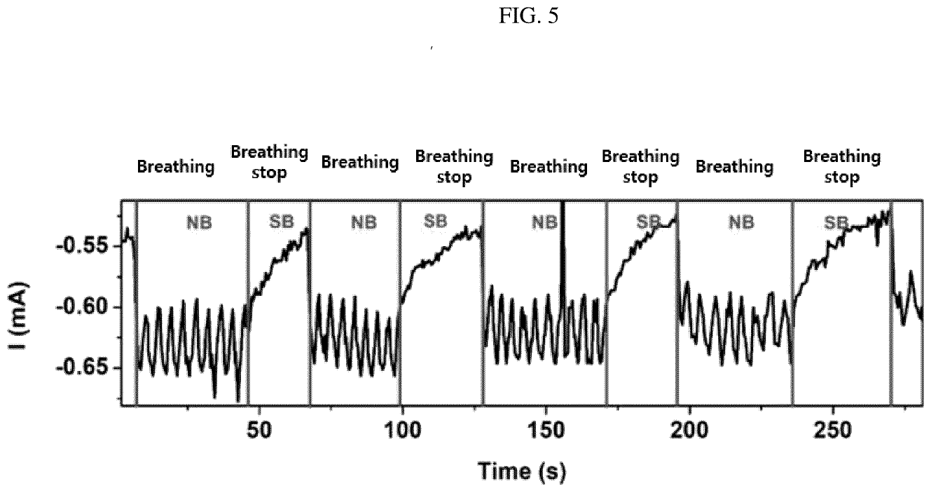

FIG. 5 is a graph showing the results obtained when measuring respiration characteristics by using a piezoresistive sensor which may be applied to a wireless respiratory monitoring device according to an embodiment of the present invention.

Referring to FIG. 5, from the results obtained when measuring respiratory characteristics by using the piezoresistive sensor, it may be seen that the current change characteristics are clearly distinguished between the respiratory section and the breathing stopping section. Therefore, if the above-mentioned piezoresistive sensor is used, the user's breathing characteristics may be measured quite accurately.

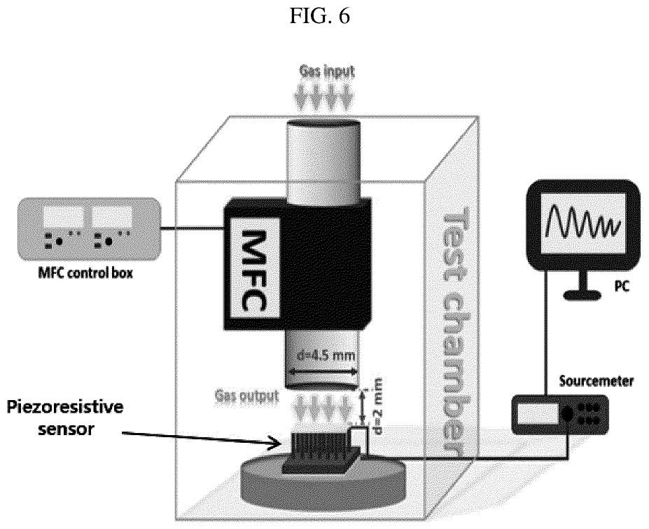

FIG. 6 is a diagram schematically showing experimental equipment for checking the sensing accuracy and sensitivity of a piezoresistive sensor that may be applied to a wireless respiratory monitoring device according to an embodiment of the present invention.

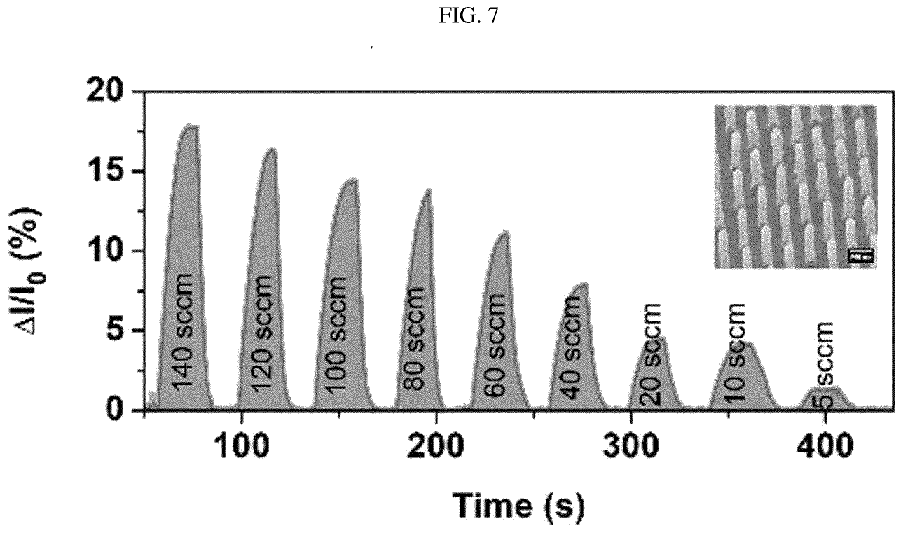

Referring to FIG. 6, under a condition that a piezoresistive sensor is placed in a test chamber, a mass flow controller (MFC) is placed above the piezoresistive sensor, and then gas is flowed toward the piezoresistive sensor through the MFC, the current change in the piezoresistive sensor was measured. At this time, the gap between the piezoresistive sensor and the gas outlet of the MFC was about 2 mm, and the diameter (inner diameter) of the gas outlet was about 4.5 mm. The change in current of the piezoresistive sensor was measured while changing the flow rate of the gas flowing (emitted) toward the piezoresistive sensor through the MFC. The results are as shown in FIG. 7.

Referring to FIG. 7, it may be seen that as the flow rate of gas flowing (emitted) toward the piezoresistive sensor changes, the amount of current change in the piezoresistive sensor changes. As the gas flow rate decreased, the amount of current change in the piezoresistive sensor decreased. In particular, it may be confirmed that the piezoresistive sensor may detect even a minute airflow of 5 sccm.

FIG. 8 is a block diagram showing a circuit configuration which may be applied to a wireless respiratory monitoring device according to an embodiment of the present invention.

Referring to FIG. 8, the wireless respiratory monitoring device according to an embodiment of the present invention may include a piezoresistive sensor 50, a circuit unit 60+65 electrically connected to the piezoresistive sensor 50, a wireless communication unit 70 electrically connected to the circuit unit 60+65 and a battery 80 for supplying power to the circuit unit 60+65 and the wireless communication unit 70.

The piezoresistive sensor 50 may be a sensor including a plurality of nanorods having a piezoresistive property, as described with reference to FIGS. 1 to 4. The circuit unit 60+65 may include a constant voltage generating circuit 60 for applying a constant voltage to the piezoresistive sensor 50, and a current measurement circuit 65 for measuring the current change of the piezoresistive sensor 50 according to the change in resistance of the plurality of nanorods. In a state where a constant voltage is applied to the piezoresistive sensor 50 by using the constant voltage generator circuit 60, the current change in the piezoresistive sensor 50 due to the user's breathing airflow may be measured by using the current measurement circuit 65.

The constant voltage generating circuit 60 may be a circuit which outputs a constant voltage of about 3V or less. The magnitude of the constant voltage may be about 0.5V to about 3V. The constant voltage generating circuit 60 may include a dedicated integrated circuit that outputs a constant voltage, or may include a circuit composed of a transistor, Zener diode, etc.

The current measurement circuit 65 may include a sensing resistor 62, an amplifier 63, and an analog-to-digital converter 64. The sensing resistor 62 is a ‘current sensing resistor’ and may be connected in series to the piezoresistive sensor 50. A potential difference may occur across the sensing resistor 62 in proportion to, for example, the amount of current flowing through the piezoresistive sensor 50. That is, the current flowing through the piezoresistive sensor 50 under constant voltage may cause a voltage drop in the sensing resistor 62. The size of the sensing resistor 62 is preferably sufficiently small compared to the piezoresistive sensor 50 to cause a current error below the noise level.

The amplifier 63 may be connected between the sensing resistor 62 and the analog-to-digital converter 64 to amplify the signal corresponding to the potential difference generated between both ends of the sensing resistor 62, and may transmit the amplified signal into an analog-to-digital converter 64. The amplifier 63 may be, for example, a differential amplification circuit using an operational amplifier. The analog-to-digital converter 64 may convert the amplified signal (analog signal) into a digital signal and transmit it to the wireless communication unit 70 connected thereto. The analog-to-digital converter 64 may have a sampling frequency of, for example, about 100 Hz or higher.

The wireless communication unit 70 may include a Bluetooth module or a wireless LAN module. The wireless communication unit 70 may be a communication module with a built-in microprocessor or microcontroller, and may include an analog-to-digital conversion circuit. The wireless communication unit 70 may use, for example, a frequency band ranging from 2400 to 2483.5 MHz.

The constant voltage generation circuit 60, the current measurement circuit 65, and the wireless communication unit 70 may be included in the circuit and communication unit 30 described in FIG. 1. Accordingly, the constant voltage generation circuit 60, the current measurement circuit 65, and the wireless communication unit 70 may be embedded and disposed within the extended body portion (e.g., 20a in FIG. 1).

The battery 80 may correspond to the battery members 40a and 40b embedded in the extended body portion (e.g., 20a/20b) in FIG. 1. As the battery 80, for example, a small rechargeable lithium ion battery, a small rechargeable lithium polymer battery, or a disposable mercury battery may be used. The battery 80 may be connected to the constant voltage generator circuit 60, the current measurement circuit 65, and the wireless communication unit 70. Furthermore, the battery 80 may be connected to the amplifier 63, the analog-to-digital converter 64, and the wireless communication unit 70 through a predetermined power circuit 85 to supply power. At this time, the power circuit 85 may be included in the circuit unit. The wireless communication unit 70 may be connected to the external terminal 90 through wireless communication and may transmit or receive signals. The external terminal 90 may be a portable terminal, for example, a mobile phone (smart phone) or a tablet PC. Alternatively, the external terminal 90 may be a laptop PC or a general computer. The wireless communication unit 70 transmits the measured signal (voltage or current signal) to the user's terminal 90 in real time and converts the measured signal into a pressure signal through a relational expression preset in the software of the terminal 90 so that respiratory may be monitored.

According to the embodiments of the present invention described above, it is possible to implement a wireless respiratory monitoring device which may be used for the diagnosis of sleep apnea, etc., and has a sensor placed inside the respiratory organ (tract) to measure respiratory airflow independently from the external environment (external airflow), while at the same time causing little discomfort even when worn while sleeping.

Most existing respiratory monitoring devices measure respiratory airflow outside the respiratory organ or estimate the amount of breathing through other biological signals. This method has the problem that measurement data may be easily distorted by the external environment (external airflow). Meanwhile, since a device for monitoring respiratory independently from the external environment uses a method covering a face as a form of a mask, it may cause discomfort to the wearer while sleeping.

However, since a wireless respiratory monitoring device according to an embodiment of the present invention measures respiratory airflow inside the nose by using an ultra-small piezoresistive sensor, and operates wirelessly with an embedded battery and wireless communication module, it may monitor respiratory independently and stably from the external environment (external airflow) without causing discomfort to the wearer. Thus, it may be effectively used to diagnose sleep apnea, and the like. In particular, since the piezoresistive sensor includes a plurality of nanorods with a piezoresistive property, and is configured to sense the wearer's respiratory characteristics by using the change in resistance caused due to deformation of the nanorods according to the change in airflow due to the wearer's (user's) breathing, excellent sensing characteristics may be secured.

Furthermore, the wireless respiratory monitoring device according to an embodiment of the present invention may not only be used to diagnose sleep apnea, but may also be applied as a healthcare device which measures respiratory to monitor sleep quality or the effect of exercise.

In this specification, the preferred embodiments of the present invention have been disclosed, and although specific terms have been used, they are only used in a general sense to easily explain the technological content of the present invention and to help understanding the present invention, and they are not used to limit the scope of the present invention. It is obvious to those having ordinary skill in the related art to which the present invention belong that other modifications based on the technological idea of the present invention may be implemented in addition to the embodiments disclosed herein. It will be understood to those having ordinary skill in the related art that in connection with wireless respiratory monitoring devices and piezoresistive sensors applied thereto according to the embodiments described with reference to FIGS. 1 to 8, various substitutions, changes, and modifications may be made without departing from the technological spirit of the present invention. Therefore, the scope of the invention should not be determined by the described embodiments, but should be determined by the technological concepts described in the claims.

1. A wireless respiratory monitoring device comprising:
a main body unit including a middle body portion having an insertion portion inserted into a user's nose, and an extended body portion extending from the middle body portion to both sides and configured to surround and hold a nosewing portion of the user;
a piezoresistive sensor which is installed on an inner surface of the insertion portion and disposed inside the user's nose, includes a plurality of nanorods having a piezoresistive property, and is configured to sense the user's breathing characteristics by using resistance changes caused by deformation of the nanorods according to airflow changes due to the user's breathing;
a circuit unit disposed within the extended body portion and electrically connected to the piezoresistive sensor;
a wireless communication unit disposed within the extended body portion and electrically connected to the circuit unit; and
a battery member disposed within the extended body portion and configured to supply power to the circuit unit and the wireless communication unit.

2. The wireless respiratory monitoring device of claim 1,
wherein the insertion portion includes a first cylindrical tube and a second cylindrical tube corresponding to the user's two nostrils,
wherein the piezoresistive sensor includes a first piezoresistive sensor installed on an inner side of the first cylindrical tube and a second piezoresistive sensor installed on an inner side of the second cylindrical tube.

3. The wireless respiratory monitoring device of claim 1, wherein the piezoresistive sensor includes:
a first electrode;
an insulating layer disposed on the first electrode to bury the plurality of nanorods from a lower portion thereof to a certain height; and
a second electrode disposed on the insulating layer to contact the plurality of nanorods,
wherein the plurality of nanorods disposed on the first electrode and substantially perpendicular to the first electrode,
wherein the plurality of nanorods include a protruding region which protrudes above the insulating layer, and the protruding region of the plurality of nanorods is configured to be deformed according to a change in airflow due to the user's breathing.

4. The wireless respiratory monitoring device of claim 3,
wherein a diameter of the plurality of nanorods is greater than or equal to 100 nm and less than 1 μm,
wherein a thickness of the insulating layer is 1 μm to 3 μm,
wherein a length of the protruding region of the plurality of nanorods is 7 μm to 14 μm.

5. The wireless respiratory monitoring device of claim 1, wherein the plurality of nanorods include at least one of silicon (Si) or zinc oxide (Zn oxide).

6. The wireless respiratory monitoring device of claim 1, wherein the circuit unit includes:
a constant voltage generating circuit for applying a constant voltage to the piezoresistive sensor; and
a current measurement circuit for measuring a change in current of the piezoresistive sensor according to a change in resistance of the plurality of nanorods.

7. The wireless respiratory monitoring device of claim 6,
wherein the current measurement circuit includes a sensing resistor, an amplifier, and an analog-to-digital converter,
wherein the sensing resistor is connected to the piezoresistive sensor,
wherein the analog-to-digital converter is connected to the wireless communication unit,
wherein the amplifier is connected between the sensing resistor and the analog-to-digital converter.

8. The wireless respiratory monitoring device of claim 1, wherein the wireless communication unit includes a Bluetooth module or a wireless LAN module.

9. The wireless respiratory monitoring device of claim 1,
wherein the extended body portion includes a first extended body portion extending from one end of the middle body portion and a second extended body portion extending from another end of the middle body portion,
wherein the battery member includes a first battery member disposed within the first extended body portion and a second battery member disposed within the second extended body portion.

10. The wireless respiratory monitoring device of claim 1, wherein at least a portion of the extended body portion and the middle body portion is made of a flexible material.

11. A wireless respiratory monitoring device comprising:
a main body unit including a middle body portion having an insertion portion inserted into a user's nose, and an extended body portion extending from the middle body portion to both sides;
a piezoresistive sensor installed on an inner surface of the insertion portion and disposed inside the user's nose, and includes a plurality of nanorods having a piezoresistive property;
a circuit unit disposed within the extended body portion and electrically connected to the piezoresistive sensor;
a wireless communication unit disposed within the extended body portion and electrically connected to the circuit unit; and
a battery member disposed within the extended body portion.

12. The wireless respiratory monitoring device of claim 11,
wherein the insertion portion includes a first cylindrical tube and a second cylindrical tube corresponding to the user's two nostrils,
wherein the piezoresistive sensor includes a first piezoresistive sensor installed on an inner side of the first cylindrical tube and a second piezoresistive sensor installed on an inner side of the second cylindrical tube.

13. The wireless respiratory monitoring device of claim 11, wherein the piezoresistive sensor includes:
a first electrode;
an insulating layer disposed on the first electrode to bury the plurality of nanorods from a lower portion thereof to a certain height; and
a second electrode disposed on the insulating layer to contact the plurality of nanorods,
wherein the plurality of nanorods disposed on the first electrode and substantially perpendicular to the first electrode,
wherein the plurality of nanorods include a protruding region which protrudes above the insulating layer, and the protruding region of the plurality of nanorods is configured to be deformed according to a change in airflow due to the user's breathing.

14. The wireless respiratory monitoring device of claim 13,
wherein a diameter of the plurality of nanorods is greater than or equal to 100 nm and less than 1 μm,
wherein a thickness of the insulating layer is 1 μm to 3 μm,
wherein a length of the protruding region of the plurality of nanorods is 7 μm to 14 μm.

15. The wireless respiratory monitoring device of claim 11, wherein the plurality of nanorods include at least one of silicon (Si) or zinc oxide (Zn oxide).

16. The wireless respiratory monitoring device of claim 11, wherein the circuit unit includes:
a constant voltage generating circuit for applying a constant voltage to the piezoresistive sensor; and
a current measurement circuit for measuring a change in current of the piezoresistive sensor according to a change in resistance of the plurality of nanorods.

17. The wireless respiratory monitoring device of claim 16,
wherein the current measurement circuit includes a sensing resistor, an amplifier, and an analog-to-digital converter,
wherein the sensing resistor is connected to the piezoresistive sensor,
wherein the analog-to-digital converter is connected to the wireless communication unit,
wherein the amplifier is connected between the sensing resistor and the analog-to-digital converter.

18. The wireless respiratory monitoring device of claim 11, wherein the wireless communication unit includes a Bluetooth module or a wireless LAN module.

19. The wireless respiratory monitoring device of claim 11,
wherein the extended body portion includes a first extended body portion extending from one end of the middle body portion and a second extended body portion extending from another end of the middle body portion,
wherein the battery member includes a first battery member disposed within the first extended body portion and a second battery member disposed within the second extended body portion.

20. The wireless respiratory monitoring device of claim 11, wherein at least a portion of the extended body portion and the middle body portion is made of a flexible material.
